(19) United States
(12) Patent Application Publication
(10) Pub. No.: US 2006/0270897 A1
(43) Pub. Date: Nov. 30, 2006

(54) Smart Sex Toys

(76) Inventor: Gregg S. Homer, Sherman Oaks, CA (US)

Correspondence Address: AKA CHAN LLP, 900 LAFAYETTE STREET, SUITE 710, SANTA CLARA, CA, 95050, US

(21) Appl. No.: 11/420,469

(22) Filed: May 25, 2006

Related U.S. Application Data
(60) Provisional application No. 60/595,014, filed on May 27, 2005.

(51) Int. Cl. A61F 5/00

(52) U.S. Cl. 600/38

(57) ABSTRACT

An arousal device has an intensity that is automatically adjusted in response to the arousal state of the user. There are three types of elements: one or more monitoring elements to collect biological data from the user relevant to his or her state of arousal; a processing element to receive the biological data and identify the appropriate intensity adjustment; and one or more output elements to deliver sensual stimulation to the user.

### CROSS-REFERENCE TO RELATED APPLICATION

The present invention claims the benefit of U.S. provisional patent application 60/595,014, filed May 27, 2005, which is incorporated by reference along with other references cited in this application.

### BACKGROUND OF THE INVENTION

The present invention relates to the field of sexual appliances and aids, and more specifically, to an aid for erotic stimulation of one's own genitals and erogenous zones.

“No other form of sexual activity has been more frequently discussed, more roundly condemned, and more universally practiced than masturbation.” Dearborn, Autoeroticism, in The Encyclopedia of Sexual Behavior (Ellis & Abarbanel eds. 1967).

Most people masturbate. A 1993 survey found that 95 percent of men and 89 percent of women had masturbated at some point in their lives. See Janus & Janus, The Janus Report on Sexual Behavior (1993). About 60 percent of men and 40 percent of women reported masturbating in the past year. See Laumann, Gagnon, et al., The Social Organization of Sexuality: Sexual Practices in the United States (1994); Michael, Gagnon, et al., Sex in America: A Definitive Survey (1994).

Despite the popularity of masturbation, its pleasures are limited by a variety of factors. Three primary factors are: (1) limited responses to stimuli from self-produced movement; (2) aversion to delayed gratification during arousal; and (3) desensitization from overstimulation. The first of these factors—limited responses to stimuli from self-produced movement—arises from the tendency of humans to filter out sensations produced by their own movements, as distinguished from those caused by changes in their environment. See Collins, Cameron, et al., Muscular Sense is Attenuated When Humans Move, 508 J. Physiol. 635-43 (1998); Milne, Aniss, et al., Reduction in Perceived Intensity of Cutaneous Stimuli During Movement: A Quantitative Study, 70 Exp. Brain Res. 569-76 (1988); Chapman, Bushnell, et al., Sensory Perception During Movement in Man, 68 Exp. Brain Res. 516-24 (1987); Angel & Malenka, Velocity-Dependent Suppression of Cutaneous Sensitivity During Movement, 7 Exp. Neurol. 266-74 (1982).

This tendency probably evolved from the fitness benefits of conserving energy by limiting defensive responses to stimuli arising from own innocuous motor actions while preserving such responses to stimuli arising from potentially deleterious external events. See Blakemore, Wolpert, et al., Why Can't You Tickle Yourself?, 11 NeuroReport 11-16 (2000). One example is the ability to distinguish movement of the eye from movement within the environment. See Helmholtz, Handbuch der Physiologischen Optik (1967); Ito, Neurophysiological Aspects of the Cerebellar Motor Control System, 7 Int'l J. Neurology, 162-76 (1970); Von Holst, Relations Between the Central Nervous System and the Peripheral Organs, 2 British J. Animal Behav. 89-94 (1954); Sperry, Neural Basis of the Spontaneous Optokinetic Response Produced by Visual Inversion, 43 J. Comp. Psychol. 482-89 (1950).

Another example is the inability to tickle oneself See Chronicle & Glover, A Ticklish Question, 39 Cortex 105-10 (2003); Simpson, Why Can't You Tickle Yourself?, 174 W. J. Medicine 425 (2001); Blakemore, Wolpert, et al., Why Can't You Tickle Yourself?, 11 NeuroReport 11-16 (2000); Blakemore, Wolpert, et al., Central Cancellation of Self-Produced Tickle Sensation, 1 Nature Neuroscience 635-40 (1998); Newman, O'Grady, et al., Pavlovian Conditioning of the Tickle Response of Human Subjects, 77 Percept. Motor Skills 779-85 (1993); Hoshikawa, Effects of Attention and Expectation of Tickle Sensation, 72 Percept. Motor Skills 27-33 (1991); Claxton, Why Can't We Tickle Ourselves?, 41 Percept. Motor Skills 335-38 (1975); Weiskranz & Darlington, Preliminary Observations on Tickling Oneself, 320 Nature 598-99 (1971).

The second of these factors—aversion to delayed gratification during arousal-refers to the difficulty men have postponing orgasm for their ideal duration. In a recent study, 152 heterosexual couples reported significantly longer ideal scripts than performance scripts for both foreplay and intercourse. See Miller & Byers, Actual and Desired Duration of Foreplay and Intercourse: Discordance and Misperceptions Within Heterosexual Couples, 41 J. Sex Res. 301-309 (2004). The inability to delay orgasm frustrates prearousal scripts. As two prominent commentators noted, “During the evolution of human sexuality, the ability to control the timing of ejaculation has become one of the most important features of couple's sexual health.” Jannini & Simonelli, Sexological Approach to Ejaculatory Dysfunction 25 Int'l J. Andrology, 317-23 (2002).

Delayed orgasm can also be helpful in the treatment is various sexual disorders. The alternative stop-start method, for example, is used in the treatment of premature ejaculation. See Kaplan, The New Sex Therapy: Active Treatment of Sexual Dysfunction (1974); Masters & Johnson, Human Sexual Inadequacy (1970). Historically, this method was limited to use with partners, but it has since been advocated during masturbation where partners are unwilling or unavailable. See Zeiss, Self-Direct Treatment of Premature Ejaculation, 46 J. Consulting Clinical Psychol., 1234-41 (1978).

The third and final factor-desensitization from overstimulation refers to the tendency of nerve tissue to be temporarily or permanently desensitized by overstimulation. Loss of cochlear sensitivity from acoustic overstimulation is well-documented. See, e.g., Flock, Flock, et al., Supporting Cells Contribute to Control of Hearing Sensitivity, 19 J. Neuroscience 4498-507 (1999); Fridberger, Van Maarseveen, et al., An In Vitro Model for Acoustic Overstimulation, 118 Acta Otolaryngol 352-61 (1998). And at least one researcher has concluded that a loss of genital sensitivity can result from over stimulation of that region during masturbation. See Sank, Traumatic Masturbatory Syndrome, 24 J. Sex. & Marital Therapy 37-42 (1998).

So-called “sex toys” or “marital aids” (collectively, “arousal devices”) have long been used in masturbation. Arousal devices in the shape of the male phallus were mentioned in the bible: “You also took the fine jewelry I gave you, the jewelry made of my gold and silver, and you made for yourself male idols and engaged in prostitution with them.” Ezekiel 16:17. The electromechanical vibrator first appeared in the 1880s as a medical instrument to bring women to orgasm in the treatment of “hysteria,” and they began appearing in erotic films in the 1920s. See Maines, Socially Camouflaged Technologies: The Case of the Electromechanical Vibrator, IEEE Technology and Society Magazine 3-11, 23 (June 1989). Although arousal devices can be valuable aids for self-gratification, none of those contemplated by the prior art resolves the three limitations on masturbations described above. Such arousal devices require operation or intervention by the user and therefore (1) constitute self-produced movement, (2) do not preclude accelerated gratification during arousal, and (3) do not preclude desensitizing overstimulation by the user.

In the case of the vibrator, desensitization is a particular problem because user sensitivity can vary greatly, so vibrators must be powerful enough to appeal to those with the least sensitivity. See Left & Israel, The Relationship Between Mode of Female Masturbation and Achievement of Orgasm in Coitus; 12 Archives Sexual Behavior 227-36 (1983); LoPiccolo & Lobitz, The Role of Masturbation in the Treatment of Orgasmic Dysfunction, 2 Archives Sexual Behavior 163-71 (1972). Although some vibrators have variable intensity settings, users' aversion to delayed gratification tend to result in an increase in intensity to the point of overstimulation once in a state of arousal. See generally Miller & Byers, supra at 301-309.

Therefore, there is a need for an arousal devices that (1) withholds control from the user, (2) is capable of delaying orgasm in accordance with the user's ideal prearousal script, and (3) sustains the minimal intensity required for arousal and, ultimately, orgasm.

### BRIEF SUMMARY OF THE INVENTION

An arousal device has an intensity that is automatically adjusted in response to the arousal state of the user. There are three types of elements: one or more monitoring elements to collect biological data from the user relevant to his or her state of arousal; a processing element to receive the biological data and identify the appropriate intensity adjustment; and one or more output elements to deliver sensual stimulation to the user.

The monitoring elements include any and all devices capable of measuring a variety of biological data, including pulse rate, blood pressure, body temperature, muscle contraction, respiration rate and intensity, and galvanic skin response. Such devices are commercially available and known to those or ordinary skill in the art.

The processing element includes any device capable of processing data. This device might include a single microchip embodied in the arousal device itself, or it might include a separate device, such as a laptop computer, desktop computer, cell phone, PDA, MP3 player, or handheld computer.

The output elements include any and all devices capable of activating the senses of the user. Such devices might deliver visual images to the ocular nerves, sounds to the auditory nerves, smells to the olfactory nerves, pheromones to the vomeronasal nerves, and/or movement or electromagnetic energy to various parts of the body, including genitals, anus, nipples, and armpits. These devices are commercially available and known to those of ordinary skill in the art.

In an embodiment, the invention is a system of masturbation including (a) means for collecting biological data from the user; (b) means for receiving and processing the biological data; and (c) means for delivering sensual stimulation to the user in response to the biological data.

In another embodiment, the invention is a system of masturbation including means for collecting biological data from the user, where the biological data are received and processed; and sensual stimulation is delivered to the user in response to the biological data.

In another embodiment, the invention is a system of masturbation including means for receiving and processing the biological data, where the biological data are collected from the user; and sensual stimulation is delivered to the user in response to the biological data.

In another embodiment, the invention is a system of masturbation including means for delivering sensual stimulation to the user in response to the biological data, where the biological data are collected from the user; and the biological data are received and processed.

In another embodiment, the invention is a method of masturbation including: (a) collecting biological data from the user; (b) receiving and processing the biological data; and (c) delivering sensual stimulation to the user in response to the biological data.

In another embodiment, the invention is a method of masturbation including collecting biological data from the user, where the biological data are received and processed; and sensual stimulation is delivered to the user in response to the biological data.

In another embodiment, the invention is a method of masturbation including receiving and processing the biological data, where the biological data are collected from the user; and sensual stimulation is delivered to the user in response to the biological data.

In another embodiment, the invention is a method of masturbation including delivering sensual stimulation to the user in response to the biological data, where the biological data are collected from the user; and the biological data are received and processed.

In another embodiment, the invention is a device for use in masturbation including: (a) at least one monitoring element to collect biological data from the user; (b) at least one processing element to receive the biological data; and (c) at least one output element to deliver sensual stimulation to the user as directed by the at least one processing element.

In another embodiment, the invention is a device for use in masturbation including at least one monitoring element to collect biological data from the user, where the biological data is received by at least one processing element; and sensual stimulation is delivered to the user by at least one output element as directed by the at least one processing element.

In another embodiment, the invention is a device for use in masturbation including at least one processing element to receive biological data, where the biological data is collected by at least one monitoring element; and sensual stimulation is delivered to the user by at least one output element as directed by the at least one processing element.

In another embodiment, the invention is a device for use in masturbation including at least one output element to deliver sensual stimulation to the user as directed by the at least one processing element, where biological data is collected from the user by the at least one monitoring element; and the biological data is received by the at least one processing element.

In another embodiment, the invention is a system including: a biological monitor device to record physiological data of a subject to which the biological monitor device monitor is coupled; a sex stimulator device to stimulate at least one erogenous zone of the subject to which the sex stimulator device is coupled; and a processor system, coupled to the biological monitor device and sex stimulator device, to alter operation of the sex stimulator device based on data received from the biological monitor device.

In the various embodiments of the invention, the biological data may include pulse rate, blood pressure, body temperature, muscle contraction, respiration rate, respiration intensity, or galvanic skin response, or any combination of these. The processing of the biological data may be by way of a microchip, laptop computer, desktop computer, cell phone, PDA, MP3 player, or handheld computer, or any combination of these. The delivering of sensual stimulation may be by visual images, sounds, smells, pheromones, movement, or electromagnetic energy, or any combination of these.

In an embodiment, no human intervention is required for the collection of the biological data. In an embodiment, no human intervention is required for the receipt and processing of the biological data. In an embodiment, no human intervention is required for the delivery of the sensual stimulation to the user.

In an embodiment, the invention is a method including: connecting a biological monitoring device to a subject; connecting a sex stimulator device to at least one erogenous zone of the subject; collecting baseline response information on the subject using the biological monitoring device; selecting a stimulation program to activate the sex stimulator device; and automatically altering operation of the sex stimulator device based on the biological monitoring device and the baseline response information.

Baseline response information may be saved for later retrieval and use. For example, the baseline response information may be used for a subsequent session with the system of the invention. The frequency or other characteristic of operation of the sex stimulator device may be altered. For example, a frequency may be reduced when the subject has become too aroused (which might be indicated by an elevated blood pressure or heart rate). Further, after a certain time after the stimulation begins, this control of reducing the frequency or other operation of the stimulation device may be canceled so that the subject can obtain an orgasm. The subject may be permitted to obtain multiple orgasms. Further, the system can detect when the subject has obtained orgasm and automatically turn off (e.g., stop the sex stimulator device or go into a cool down mode of operation). The device may automatically turn off after a specific number of orgasms, not just one.

Other objects, features, and advantages of the present invention will become apparent upon consideration of the following detailed description and the accompanying drawings, in which like reference designations represent like features throughout the figures.

### BRIEF DESCRIPTION OF THE DRAWINGS

FIG. 1 represents the prior art, where the arousal device consists of a vibrator in the shape of the male phallus, and a female user controls its placement and intensity.

FIG. 2 represents a specific embodiment of the invention, where the arousal device includes (1) monitor elements that measure pulse rate, blood pressure, and muscle contraction, (2) a processing element consisting of a laptop computer, and (3) output elements that deliver erotic visual images to the eyes and vibration to the nipples, genitals, and anus.

FIG. 3 shows a processing element of a system of the invention.

FIG. 4 shows a simplified system block diagram of a processing element.

### DETAILED DESCRIPTION OF THE INVENTION

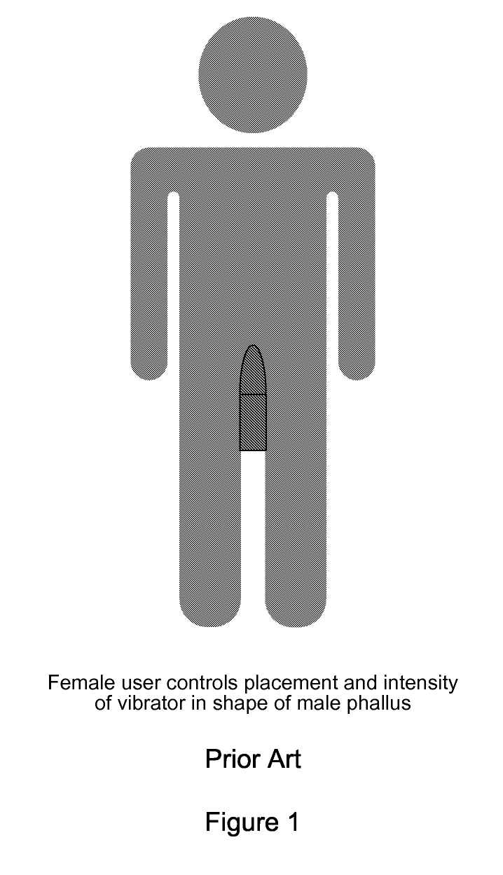

FIG. 1 represents the prior art, where the arousal device consists of a vibrator in the shape of the male phallus, and a female user controls its placement and intensity. Prior art is discussed in more detail in the Background of the Invention section of this patent application.

The present invention is an arousal device, the intensity of which is automatically adjusted in response to the arousal state of the user. There are three types of elements: one or more monitoring elements to collect biological data from the user relevant to his or her state of arousal; a processing element to receive the biological data and identify the appropriate intensity adjustment; and one or more output elements to deliver sensual stimulation to the user.

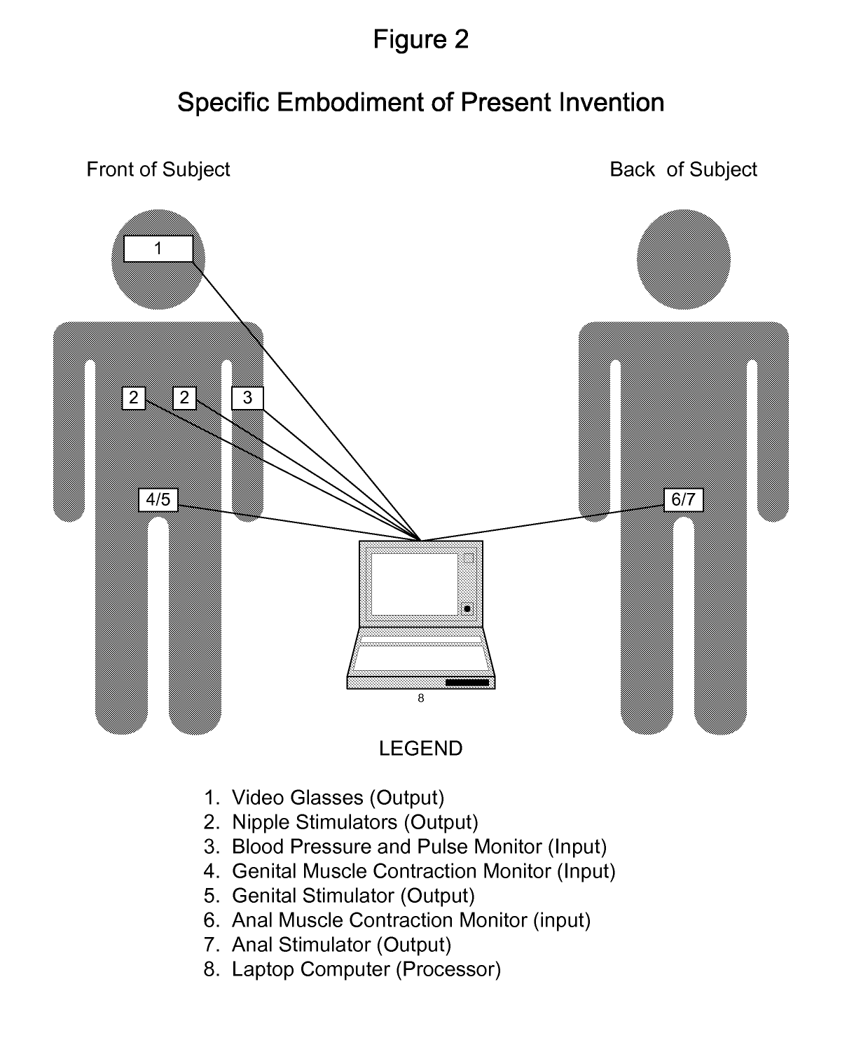

FIG. 2 represents a specific embodiment of the invention, where the arousal device includes (1) monitor elements that measure pulse rate, blood pressure, and muscle contraction, (2) a processing element consisting of a laptop computer, and (3) output elements that deliver erotic visual images to the eyes and vibration to the nipples, genitals, and anus.

The monitoring elements include any and all devices capable of measuring a variety of biological data, including pulse rate, blood pressure, body temperature, muscle contraction, respiration rate and intensity, and galvanic skin response.

This specific embodiment has a multitude of biological monitor inputs including blood pressure and pulse monitor 3, genital muscle contraction monitor 4, and anal muscle contraction monitor 6. These are shown merely as examples of possible inputs. There may be other biological monitor inputs than the ones specifically listed. A system may include all the biological monitor inputs shown and other biological monitor inputs not specifically listed. Further, a system of the invention may include any one biological monitor input or any combination of biological monitor input. For example, a system may include only the anal muscle contraction monitor. A system may include the blood pressure and pulse monitor and genital muscle contraction monitor, but not anal muscle contraction monitor.

Some biological monitor devices are commercially available and known to those or ordinary skill in the art. Examples of such devices include the following: (1) the Brackmann II EMG System from WR Medical Electronics Co. (Stillwater, Minn.), which measures muscle contractions; (2) the GSR Sensor from Qubit Systems Inc. (Kingston, Ontario, Canada), which measures galvanic skin response; and (3) the Biomonitor from Electronic Engineering Corporation (Tamilnadu, India), which tracks pulse rate and rhythm, respiration rate and rhythm, body temperature, blood pressures, and heart sounds. Any of these or similar devices may be used in a system of the invention.

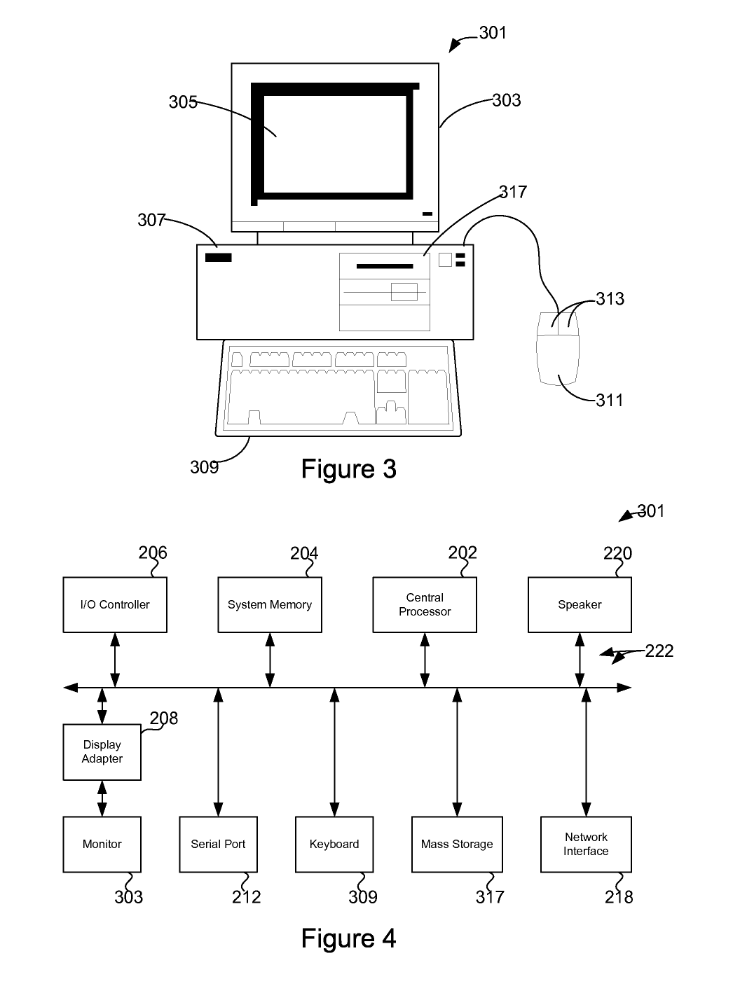

The processing element 8 includes any device capable of processing data. This device might consist of a single microchip embodied in the arousal device itself, or it might consist of a separate device, such as a laptop computer, desktop computer, cell phone, PDA, MP3 player, or handheld computer. A variety of processing elements are commercially available and known to those of ordinary skill in the art. Examples of processing elements include: (1) the Sony VAIO T350P/S Notebook VGN-T350P/S from Sony Corporation (Tokyo, Japan); and (2) the HP iPaq Pocket PC h6315 from Hewlett-Packard Company (Palo Alto, Calif.). More details of the processing element are provided in FIGS. 3 and 4 and the accompanying discussion below.

The output elements include any and all devices capable of activating the senses of the user. Such devices might deliver visual images to the ocular nerves, sounds to the auditory nerves, smells to the olfactory nerves, pheromones to the vomeronasal nerves, and/or movement or electromagnetic energy to various parts of the body, including genitals, anus, nipples, and armpits.

This specific embodiment has a multitude of output elements including video glasses 1, nipple stimulators 2, genital stimulator 5, and anal stimulator 7. These are shown merely as examples of possible outputs. There may be other outputs than the ones specifically listed. A system may include all the outputs shown and also other outputs not specifically listed. Further, a system of the invention may include any one output element or any combination of output elements. For example, a system may include only the genital stimulator. A system may include only the video glasses and anal stimulator.

Some of these devices are commercially available and known to those of ordinary skill in the art. Examples of these devices include: (1) female genital vibrators, such as Gina Lynn's Vibrating Adventure Arouser from California Exotic Novelties, LLC (Chino, Calif.); (2) male genital suction devices, such as Robo Suck II from Doc Johnson Toys International (North Hollywood, Calif.); (3) male and female anal vibrators, such as the Anal Invader from Doc Johnson Toys International (North Hollywood, Calif.); and (4) male and female nipple vibrators, such as the Devinn Lane's Waterproof Nipple Arousers from California Exotic Novelties, LLC (Chino, Calif.). Any of these or similar devices may be used in a system of the invention.

FIGS. 3 and 4 show an example of a processing element of a system of the invention. In an embodiment, the invention includes software that executes on a computer workstation system, such as shown in FIG. 3. FIG. 3 shows a computer system 301 that includes a monitor 303, screen 305, cabinet 307, keyboard 309, and mouse 311. Mouse 311 may have one or more buttons such as mouse buttons 313. Cabinet 307 houses familiar computer components, some of which are not shown, such as a processor, memory, mass storage devices 317, and the like.

Mass storage devices 317 may include mass disk drives, floppy disks, magnetic disks, optical disks, magneto-optical disks, fixed disks, hard disks, CD-ROMs, recordable CDs, DVDs, recordable DVDs (e.g., DVD-R, DVD+R, DVD-RW, DVD+RW, HD-DVD, or Blu-ray Disc), flash and other nonvolatile solid-state storage (e.g., USB flash drive), battery-backed-up volatile memory, tape storage, reader, and other similar media, and combinations of these.

A computer-implemented version of the invention may be embodied using, or reside on, computer readable medium. A computer-readable medium may include any medium that participates in providing instructions to one or more processors for execution. Such a medium may take many forms including, but not limited to, nonvolatile, volatile, and transmission media. Nonvolatile media includes, for example, flash memory or optical or magnetic disks. Volatile media includes static or dynamic memory, such as cache memory or RAM. Transmission media includes coaxial cables, copper wire, fiber optic lines, and wires arranged in a bus. Transmission media can also take the form of electromagnetic, radio frequency, acoustic, or light waves, such as those generated during radio wave and infrared data communications.

For example, a binary, machine-executable version, of the software of the present invention may be stored or reside in RAM or cache memory, or on mass storage device 317. The source code of the software of the present invention may also be stored or reside on mass storage device 317 (e.g., hard disk, magnetic disk, tape, or CD-ROM). As a further example, code of the invention may be transmitted via wires, radio waves, or through a network such as the Internet.

FIG. 4 shows a system block diagram of computer system 301 used to execute software of the present invention. As in FIG. 3, computer system 301 includes monitor 303, keyboard 309, and mass storage devices 317. Computer system 301 further includes subsystems such as central processor 202, system memory 204, input/output (I/O) controller 206, display adapter 208, serial or universal serial bus (USB) port 212, network interface 218, and speaker 220. The invention may also be used with computer systems with additional or fewer subsystems. For example, a computer system could include more than one processor 202 (i.e., a multiprocessor system) or the system may include a cache memory. The processor may be a dual core or multicore processor, where there are multiple processor cores on a single integrated circuit.

Arrows such as 222 represent the system bus architecture of computer system 301. However, these arrows are illustrative of any interconnection scheme serving to link the subsystems. For example, speaker 220 could be connected to the other subsystems through a port or have an internal connection to central processor 202. Computer system 301 shown in FIG. 3 is but an example of a computer system suitable for use with the present invention. Other configurations of subsystems suitable for use with the present invention will be readily apparent to one of ordinary skill in the art.

Computer software products may be written in any of various suitable programming languages, such as C, C++, C#, Pascal, Fortran, Perl, MatLab (from MathWorks, Inc.), SAS, SPSS, Java, JavaScript, and AJAX. The computer software product may be an independent application with data input and data display modules. Alternatively, the computer software products may be classes that may be instantiated as distributed objects. The computer software products may also be component software such as Java Beans (from Sun Microsystems) or Enterprise Java Beans (EJB from Sun Microsystems).

An operating system for the system may be one of the Microsoft Windows® family of operating systems (e.g., Windows 95, 98, Me, Windows NT, Windows 2000, Windows XP, Windows XP x64 Edition, Windows Vista, Windows CE, Windows Mobile), Linux, HP-UX, UNIX, Sun OS, Solaris, Mac OS X, Alpha OS, AIX, IRIX32, or IRIX64, or combinations of these. Other operating systems may be used. A computer in a distributed computing environment may use a different operating system from other computers.

Furthermore, the computer may be connected to a network and may interface to other computers using this network. Furthermore, the network may be an intranet, internet, or the Internet, among others. The network may be a wired network (e.g., using copper), telephone network, packet network, an optical network (e.g., using optical fiber), or a wireless network, or any combination of these. For example, data and other information may be passed between the computer and components (or steps) of a system of the invention using a wireless network using a protocol such as Wi-Fi (IEEE standards 802.11, 802.11a, 802.11b, 802.11e, 802.11g, 802.1 μl, and 802.1 in, just to name a few examples). For example, signals from a computer may be transferred, at least in part, wirelessly to components of the system or other computers.

In an embodiment of the invention, the biological monitor inputs and output elements are connected to the appropriate locations a person's body. They are also connected to the processor system via a universal serial bus (USB) or other peripheral port, such as Firewire, serial, parallel, ethernet, modem, and many others. The user selects a session time. Before activating any of the sex toys, the processor system collects and stores baseline data, such as pulse rate, blood pressure, and body temperature, from the user (or subject) through the biological monitors.

It then begins activating the output elements based on a predetermined program of speed, intensity, and location in order to establish a personalized stimulation protocol for the subject. Once the protocol is established, a processor element begins operating the output elements in accordance with the protocol. As the session progresses, the processor system continues to collect data from the subject and modify the protocol in order to achieve the desired state. One desired state may be a Tantric state, which is the state of sexual arousal just prior to orgasm, and to maintain that state until the expiration of the subject's preselected session time. At that point, the processor system can stimulate the subject to the point of orgasm or simply end the session without orgasm. The subject's session data and protocol are then stored for use in future sessions. It can also be used to assist couples in improving their sexual relations by identifying optimal speeds, intensities, locations, and timing of stimulation.

In an embodiment, the arousal device includes a battery-operated vibrator in the shape of a male phallus. This device is equipped with various monitors that measure pulse rate, blood pressure, body temperature, and muscle contraction. A female user inserts the device into her vagina and turns on the power. The monitors are in contact with the tissues lining the user's vaginal wall and collect a baseline measurement of the user's biographical data. Vibration is a wave form, so its intensity is typically expressed in terms of its frequency and amplitude. Frequency is expressed in either hertz or in “beats” per minute, and amplitude is expressed as the range of movement in each vibratory beat. Let us assume that the present embodiment has a variable frequency and a fixed amplitude of 2 millimeters.

The device begins to vibrate at an extremely low frequency, e.g., 60 beats per minute, and gradually accelerates until initial arousal is achieved. Anytime user arousal ceases to increase, the frequency of vibration is increased, but always in small increments, e.g., 10 percent. The range of vibratory frequency among most commercially available personal vibrators is 5000-6000 beats per minute. In some cases, the vibratory frequency of the present embodiment might equal or exceed this range. In most cases, however, a far lower frequency will be required to achieve arousal and orgasm. This gradual increase of frequency continues until the user achieves orgasm.

In another embodiment, the arousal device is far more complex. The monitor elements attach to various parts of the body and measure pulse rate, blood pressure, body temperature, muscle contraction, respiration rate and intensity, and galvanic skin response. The processing element consists of a laptop computer. The output elements deliver heat, vibration, suction, and mild electrical current to the genitals, anus, and nipples. The monitor and output elements attach to the laptop computer through USB ports in the laptop.

A male user sets the arousal device to delay orgasm for a period of 20 minutes. He attaches the various monitor and output devices to the designated areas of his body and activates the arousal device. Biological data are collected by the monitoring devices and delivered to the laptop.

A proprietary software program analyses the data and identifies arousal trends. It computes the ideal arousal protocol for the user and operates the various output devices accordingly. Throughout the 20-minute period selected by the user, the output elements are activated at an intensity calculated to stimulate the user to a state of arousal just prior to orgasm, then reduce the intensity of output until the user's arousal state falls to some intermediate point. As the end of the 20-minute period approaches, the arousal device gradually increases the intensity of the output elements until the user achieved orgasm.

In one embodiment of the present invention, the subject is a 30-year-old female. She uses pulse rate, blood pressure, body temperature, and vaginal muscle contraction monitors as biological monitors; a PDA as the processor system; and a genital vibrator, nipple suction, and an anal motion device as output elements. The subject installs the proprietary software on the PDA, connects the biological monitors and output elements to the appropriate locations on her body, and connects the biological monitors and output elements to the processor element via a USB port.

The subject then selects a session time of 20 minutes (with orgasm), assumes a comfortable seated or reclining position, and initiates the processor system's software program. The processor system collects the subject's baseline data through the biological monitors. The processor system then begins activating the output elements based on a standardized speed, intensity, and location established by the software program. It collects data from the subject through the biological monitors, analyzes the data, and establishes a personalized stimulation protocol for the subject. The personalized protocol then assumes control of the output elements.

The nipple stimulator is activated first. It applies gentle suction to the subject's nipples. About 15 seconds later, the nipple stimulator is suspended, and the anus stimulator is activated at a minimal level of movement. After 15 more seconds, the nipple and anal alternate at five second intervals. Finally, the genital stimulator is activated at an almost imperceptible vibrational frequency. The toys continue to operate in accordance with the personalized protocol.

Meanwhile, the processor continues to gather data through the biological monitors and modify the personalized protocol required to maintain the highest possible level of arousal without inducing orgasm until the expiration of the subject's preselected 20-minute session time, at which point the processor system stimulates the subject to the point of orgasm. The subject's session data and protocol are stored for use in future sessions. The processor system also prints out a report on the subject's session, identifying the optimal speeds, intensities, locations, and timing of stimulation, which the subject may deliver to her husband for review.

In another embodiment, the subject is a 65-year-old male. The biological monitors selected by the subject measure anal muscle contraction, respiration rate and intensity, and galvanic skin response. The subject's processor system is a laptop computer. The selected sex stimulators deliver mild electrical current to the nipples, suction to the penis, vibration to the anus, and erotic audio-visual images and sounds to his eyes and ears through 3-D video glasses. The sounds and images are synchronized with the sensations delivered by the other sex stimulators. For example, when a character in the images kisses the subject's nipples, the nipple sex toy is activated. The audio-visual images and sounds are part of the subject's personalized protocol and are modified based on biological data gathered through the subject's biological monitors.

The subject installs the proprietary software on the laptop, connects the biological monitors and sex stimulators to the appropriate locations on his body, and connects the biological monitors and sex stimulators to the processor system via a USB port. The subject then selects a session time of 10 minutes (without orgasm), assumes a comfortable seated or reclining position, and initiates the processor system's software program. The processor system collects the subject's baseline data through the biological monitors.

The processor system then activates and modifies the sex stimulators in the manner described in the previous specific embodiment until the expiration of the subject's 10-minute session time, at which point the processor system shuts down the sex toys without orgasm. The subject's session data and protocol are stored for use in future sessions.

Operation of arousal devices may be modified using different technologies. For example, in a computer-implemented method, images appearing on a display device are influenced by the manner in which the user touches the display screen. In an interactive virtual sexual stimulation system, a first person is located at a first user interface and can moderate the arousal of a second person located at a second user interface by controlling various arousal devices located at the second user interface. And further, sensors on the first person's body can control the arousal devices located at the second user interface, allowing for the possibility of remote sexual intimacy. However, none of these approaches provides the features and benefits of the present invention. Each anticipates some human intervention by either the user or another party. The present invention, by contrast, is entirely automated, responding solely to the biological data collected from the user, with no human intervention required.

An arousal device may be activated by biofeedback generated signals. However, biofeedback is different from the invention. First, “biofeedback” is “the use of electronic monitoring of a normally automatic bodily function in order to train a person to acquire voluntary control of that function.” The New Shorter Oxford English Dictionary 230 (Brown ed. 1993). In other words, biofeedback collects biological data from a given bodily function of the user and delivers those data to the user so that he or she can learn to control that bodily function. Second, an arousal device activated by a biofeedback generated signal will not have adjustment of the arousal device in response to the user's arousal level.

The present invention, by contrast, collects biological data from a given bodily function of the user and delivers those data directly to the arousal device so that the arousal device can control that bodily function. Biofeedback requires human intervention. The present invention does not.

In the invention, the processing unit alters the signal to the stimulation or output elements to they vary their output. The altering of the signal and the output may be based on an algorithm embodied using software code or in firmware. In an embodiment, the altering may not involve interaction or intervention of either the user or another party. For example, the output element may vary its vibration frequency or intensity based on the monitored input signal, without requiring user intervention or user interaction such as turning a knob or flipping a switch.

In an embodiment, the level of the signal to the output element (where the signal controls the operation of the output element) may be varied without interaction of the user.

For safety and comfort, there may be a maximum level of the signal to the output element, so regardless of the input signal being monitored, a maximum level will not be exceeded. There may be different versions of the system (or software or firmware) with different maximum levels.

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

1. A system of masturbation comprising:
(a) means for collecting biological data from the user;
(b) means for receiving and processing the biological data; and
(c) means for delivering sensual stimulation to the user in response to the biological data.

2. A system of masturbation comprising:
means for collecting biological data from the user,
wherein the biological data are received and processed, and
sensual stimulation is delivered to the user in response to the biological data.

3. A system of masturbation comprising:
means for receiving and processing the biological data,
wherein the biological data are collected from the user, and
sensual stimulation is delivered to the user in response to the biological data.

4. A system of masturbation comprising:
means for delivering sensual stimulation to the user in response to the biological data,
wherein the biological data are collected from the user, and
the biological data are received and processed.

5. The system of claim 1 wherein the means for collecting biological data from the user comprises at least one of pulse rate, blood pressure, body temperature, muscle contraction, respiration rate, respiration intensity, or galvanic skin response.

6. The system of claim 1 wherein the means for receiving and processing the biological data comprises at least one of a microchip, laptop computer, desktop computer, cell phone, PDA, MP3 player, or handheld computer.

7. The system of claims claim 1 wherein the means for delivering sensual stimulation to the user comprises at least one of visual images, sounds, smells, pheromones, movement, or electromagnetic energy.

8. The system of claim 7 wherein the sensual stimulation is delivered to at least one of the genitals, anus, nipples, or armpits of the user.

9. The system of claim 1 wherein no human intervention is required for the collection of the biological data.

10. The system of claim 1 wherein no human intervention is required for the receipt and processing of the biological data.

11. The system of claim 1 wherein no human intervention is required for the delivery of the sensual stimulation to the user.

12. A method of masturbation comprising:
(a) collecting biological data from the user;
(b) receiving and processing the biological data; and
(c) delivering sensual stimulation to the user in response to the biological data.

13. A method of masturbation comprising:
collecting biological data from the user,
wherein the biological data are received and processed, and
sensual stimulation is delivered to the user in response to the biological data.

14. A method of masturbation comprising:
receiving and processing the biological data,
wherein the biological data are collected from the user, and
sensual stimulation is delivered to the user in response to the biological data.

15. A method of masturbation comprising:
delivering sensual stimulation to the user in response to the biological data,
wherein the biological data are collected from the user, and
the biological data are received and processed.

16. The method of claim 12 wherein the biological data collected from the user comprise at least one of pulse rate, blood pressure, body temperature, muscle contraction, respiration rate, respiration intensity, or galvanic skin response.

17. The method of claim 12 wherein the biological data are received and processed on at least one of a microchip, laptop computer, desktop computer, cell phone, PDA, MP3 player, or handheld computer.

18. The method of claims claim 12 wherein the sensual stimulation delivered to the user comprise at least one of visual images, sounds, smells, pheromones, movement, or electromagnetic energy.

19. The method of claim 18 wherein the sensual stimulation is delivered to at least one of the genitals, anus, nipples, or armpits of the user.

20. The system of claim 12 wherein no human intervention is required for the collection of the biological data.

21. The system of claim 12 wherein no human intervention is required for the receipt and processing of the biological data.

22. The system of claim 12 wherein no human intervention is required for the delivery of the sensual stimulation to the user.

23. A device for use in masturbation comprising:
(a) at least one monitoring element to collect biological data from the user;
(b) at least one processing element to receive the biological data; and
(c) at least one output element to deliver sensual stimulation to the user as directed by the at least one processing element.

24. A device for use in masturbation comprising:
at least one monitoring element to collect biological data from the user,
wherein the biological data is received by at least one processing element; and
sensual stimulation is delivered to the user by at least one output element as directed by the at least one processing element.

25. A device for use in masturbation comprising:
at least one processing element to receive biological data,
wherein the biological data is collected by at least one monitoring element, and
sensual stimulation is delivered to the user by at least one output element as directed by the at least one processing element.

26. A device for use in masturbation comprising:
at least one output element to deliver sensual stimulation to the user as directed by the at least one processing element,
wherein biological data is collected from the user by the at least one monitoring element, and
the biological data is received by the at least one processing element.

27. The method of claim 23 wherein the biological data collected from the user comprise at least one of pulse rate, blood pressure, body temperature, muscle contraction, respiration rate, respiration intensity, or galvanic skin response.

28. The method of claim 23 wherein the biological data are received and processed on at least one of a microchip, laptop computer, desktop computer, cell phone, PDA, MP3 player, or handheld computer.

29. The method of claim 23 wherein the sensual stimulation delivered to the user comprise at least one of visual images, sounds, smells, pheromones, movement, or electromagnetic energy.

30. The method of claim 29 wherein the sensual stimulation is delivered to at least one of the genitals, anus, nipples, or armpits of the user.

31. The system of claim 23 wherein no human intervention is required for the collection of the biological data.

32. The system of claim 23 wherein no human intervention is required for the receipt and processing of the biological data.

33. The system of claim 23 wherein no human intervention is required for the delivery of the sensual stimulation to the user.

34. A system comprising:
a biological monitor device to record physiological data of a subject to which the biological monitor device monitor is coupled;
a sex stimulator device to stimulate at least one erogenous zone of the subject to which the sex stimulator device is coupled; and
a processor system, coupled to the biological monitor device and sex stimulator device, to alter operation of the sex stimulator device based on data received from the biological monitor device.

35. A method comprising:
connecting a biological monitoring device to a subject;
connecting a sex stimulator device to at least one erogenous zone of the subject;
collecting baseline response information on the subject using the biological monitoring device;
selecting a stimulation program to activate the sex stimulator device; and
automatically altering operation of the sex stimulator device based on the biological monitoring device and the baseline response information.

36. The method of claim 35 further comprising:
saving the baseline response information for later retrieval and use.

37. The method of claim 35 wherein the automatically altering operation comprises:
reducing a frequency of operation of the sex stimulator device when the biological monitoring device detects the subject has become too aroused.

38. The method of claim 37 wherein after a specific time period after activating the sex stimulator device cancelling the reducing a frequency of operation of the sex stimulator device when the biological monitoring device detects the subject has become too aroused, whereby the subject is permitted to orgasm.

39. The method of claim 37 wherein the subject is too aroused when the heart rate is above a certain level.

40. The method of claim 35 wherein the automatically altering operation comprises:
increasing a frequency of operation of the sex stimulator device when the biological monitoring device detects the subject is not sufficiently aroused.

41. The method of claim 35 wherein when the biological monitoring device detects the subject has achieved an orgasm, stopping the sex stimulator device.

42. The method of claim 35 wherein when the biological monitoring device detects the subject has achieved a specific number of orgasms, stopping the sex stimulator device.
